(19) United States
(12) Patent Application Publication
(10) Pub. No.: US 2024/0113534 A1
(43) Pub. Date: Apr. 4, 2024

(54) WAKE-ABLE ELECTRONIC DEVICE AND METHODS FOR WAKING THEREOF

(71) Applicant: The Alfred E. Mann Foundation for Scientific Research, Valencia, CA (US)

(72) Inventors: Jeffrey A. Brydle, Pasadena, CA (US); Brian Shelton, Ventura, CA (US); Brian Dearden, Pasadena, CA (US); Rongqing Dai, Valencia, CA (US)

(21) Appl. No.: 18/477,435

(22) Filed: Sep. 28, 2023

Related U.S. Application Data
(60) Provisional application No. 63/412,245, filed on Sep. 30, 2022.

(51) Int. Cl. H02J 7/00; A61N 1/36; G01R 31/396; H02J 50/12

(52) U.S. Cl. CPC H02J 7/0031; A61N 1/36; G01R 31/396; H02J 7/0044; H02J 50/12

(57) ABSTRACT

An electronic device, includes: a battery; a microcontroller configured to receive power from the battery along a first path comprising a first battery switch configured to change between an opened configuration and a closed configuration; and a receiver coil configured, in response to being positioned in a changing magnetic field, to generate an alternating electric current and to change, via the alternating electric current, the first battery switch from the opened configuration to the closed configuration.

### CROSS-REFERENCE TO RELATED APPLICATION

The present application claims priority to and the benefit of U.S. Provisional Application No. 63/412,245, filed Sep. 30, 2022, entitled “WAKE-ABLE ELECTRONIC DEVICE AND METHODS FOR WAKING THEREOF”, the entire content of which is incorporated herein by reference.

### BACKGROUND

### 1. Field

The present disclosure relates to wake-able electronic devices and methods for waking such electronic devices.

### 2. Description of the Related Art

Some electronic devices, for example, implantable medical devices, may include an internal battery and other components that are powered by the internal battery. There may be one or more times during the lifetime of such electronic devices where the electronic device is not in use for a significant period of time, for example, at least part of the time between when the electronic device is manufactured and when it is sold to a customer. However, unless at least some of the components of the electronic device are electrically disconnected from the battery, there is a risk that such components may continue to draw power from the battery and drain the battery. If the voltage of the battery drops below a minimal threshold, it may be difficult, impossible, or potentially hazardous to try to recharge the battery.

### SUMMARY

The present disclosure relates to various embodiments of an electric device. In one embodiment, the electronic device includes a battery; a microcontroller configured to receive power from the battery along a first path comprising a first battery switch configured to change between an opened configuration and a closed configuration; and a receiver coil configured, in response to being positioned in a changing magnetic field, to generate an alternating electric current and to change, via the alternating electric current, the first battery switch from the opened configuration to the closed configuration.

The present disclosure relates to various embodiments of a system. In one embodiment, the system includes: a battery, and a microcontroller configured to receive power from the battery along a first path including a first battery switch configured to be changed between an opened configuration and a closed configuration; and an external charger configured to wirelessly charge the battery and to change the first battery switch from the opened configuration to the closed configuration, wherein the electronic device is configured, in response to the first battery switch being changed from the opened configuration to the closed configuration by the external charger such that power is provided from the battery to the microcontroller, to: maintain, by the microcontroller, the first battery switch in the closed configuration for a set time period, and cease to maintain, by the microcontroller and in response to not receiving a communication from an external device within the set time period, the first battery switch in the closed configuration.

The present disclosure relates to various embodiments of a method of operating an implantable device. In one embodiment, the method includes taking the implantable device out of a shelf mode, in which a microcontroller of the implantable device is electrically disconnected from a battery of the implantable device, and into a normal mode, in which the microcontroller is electrically connected to the battery of the implantable device, in response to a receiver coil of the implantable device receiving a charge field from a first external device and via an electric current generated by the receiver coil.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter.

### BRIEF DESCRIPTION OF THE DRAWINGS

The drawings, together with the specification, illustrate example embodiments of the present disclosure.

FIG. 1A is a schematic view of a system according to some embodiments.

FIG. 1B is a schematic block diagram of an electronic device of the system of FIG. 1A according to some embodiments.

FIG. 1C is a circuit diagram showing at least some components of the electronic device of the stimulation system of FIG. 1A according to some embodiments.

FIG. 2 is a shelf mode method flow diagram for placing an electronic device in a shelf mode.

FIG. 3 is a normal mode method flow diagram for bringing an electronic device out of the shelf mode.

### DETAILED DESCRIPTION

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section, without departing from the spirit and scope of the present disclosure.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises,” “comprising,” “includes,” and “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element is referred to as being “on”, “connected to”, “coupled to”, or “adjacent to” another element, it can be directly on, connected to, coupled to, or adjacent to the other element, or one or more intervening elements may be present. In contrast, when an element is referred to as being “directly on,” “directly connected to,” “directly coupled to,” or “immediately adjacent to” another element, there are no intervening elements present.

As used herein, the term “substantially” and similar terms are used as terms of approximation and not as terms of degree, and are intended to account for the inherent deviations in measured or calculated values that would be recognized by those of ordinary skill in the art. Also, the term “about” and similar terms, when used herein in connection with a numerical value or a numerical range, are inclusive of the stated value and mean within an acceptable range of deviation for the particular value as determined by one of ordinary skill in the art, considering the measurement in question and the error associated with measurement of the particular quantity (e.g., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Non-limiting and non-exhaustive embodiments of electronic devices, systems including an electronic device, and methods of operating electronic devices will now be described herein with reference to the drawings.

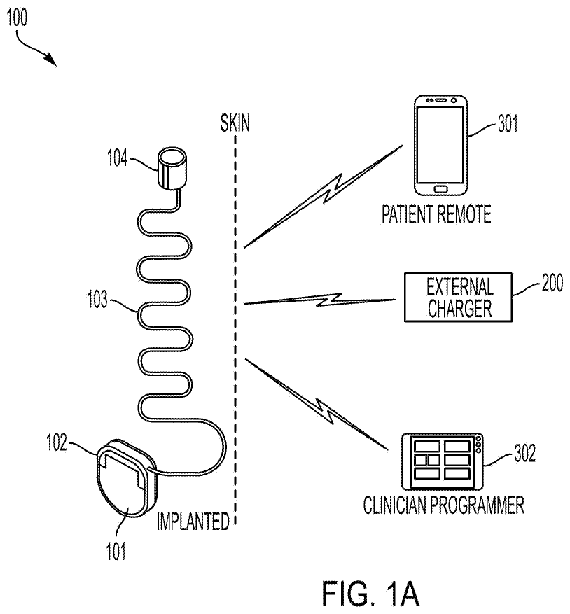

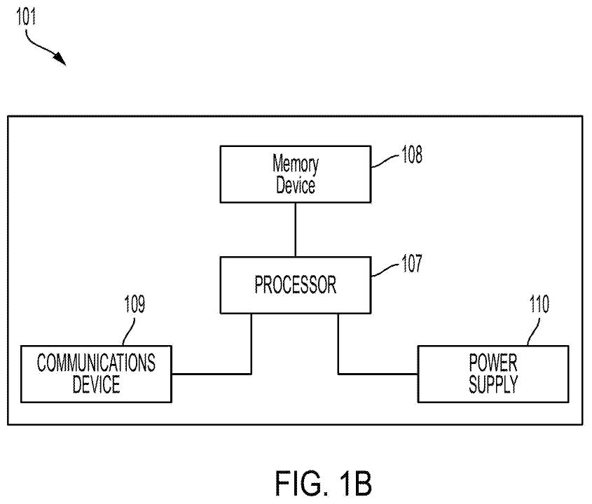

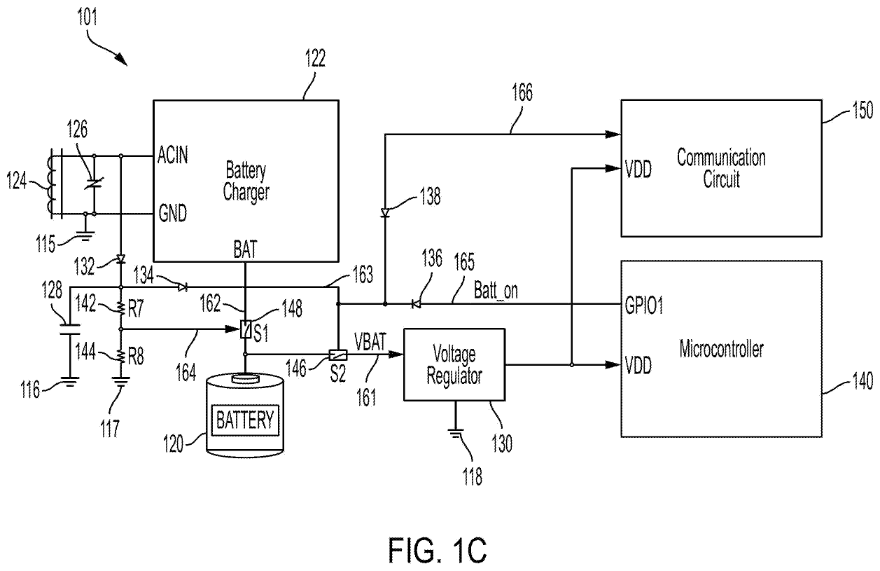

FIG. 1A is a schematic view of a system 100 according to some embodiments. FIG. 1B is a schematic block diagram of an electronic device 101 of the system 100 of FIG. 1A according to some embodiments. FIG. 1C is a circuit diagram showing at least some components of the electronic device 101 of the system 100 of FIG. 1A according to some embodiments.

Referring to FIG. 1A, in some embodiments, the system 100 is a medical device system, such as a stimulation system. In the depicted embodiment, the system 100 is a stimulation system, and the system 100 may be referred to herein as a stimulation system. However, it will be understood that the system 100 is not limited thereto, and that the teachings disclosed herein regarding the stimulation system 100 may be applied to other systems. The stimulation system 100 may include an electronic device 101. The electronic device 101 may be an implantable medical device, such as an implantable pulse generator (IPG) configured to be implanted in a person or living being and to provide stimulation, for example, to biological tissue in proximity with the IPG. In the depicted embodiment, the electronic device 101 is depicted as an IPG, and the electronic device 101 may be referred to herein as an IPG. However, it will be understood that the electronic device 101 is not limited thereto, and that the teachings disclosed herein regarding the IPG 101 may be applied to other electronic devices.

The IPG 101 may include a housing 102 (e.g., a case) and an electrical lead 103 electrically coupled to the housing 102 and including an electrode 104 at an end of the electrical lead 103 distal to the housing 102. The IPG 101 may be configured to provide stimulation by controllably driving the electrode 104 with an electric current provided from a driver in the housing 102 and through the electrical lead 103. Although the depicted embodiment depicts the IPG 101 as including one electrical lead 103 that includes one electrode 104, the present disclosure is not limited thereto. In some embodiments, the IPG may include one or more electrical leads electrically coupled to the housing, and each of the one or more electrical leads may include one or more electrodes and/or one or more electrical conductors (e.g., conductor coils) respectively positioned at any suitable position along the electrical lead and arranged with each other on the electrical lead in any suitable manner. For example, an electrical lead may include two electrodes, one of the two electrodes being positioned at a distal end of the electrical lead and the other one of the two electrodes being positioned half-way along the electrical lead. The electrical conductor(s) may be configured to generate a changing magnetic field when driven with an alternating current, and the magnetic field may induce electric currents in the biological tissue in proximity to the electrical conductor(s).

The housing 102 may be configured to contain at least some components of the IPG 101 (as described in more detail below) and substantially electrically insulate the components contained within the housing 102 from the surrounding tissue. The housing 102 may have one or more openings configured to receive at least part of a proximal end of the electrical lead 103.

The stimulation system 100 may include an external charger 200 configured to wirelessly charge or power (e.g., via induction) the IPG 101. For example, the IPG 101 may be configured such that the IPG 101 can store energy wirelessly received from the external charger 200 in a rechargeable battery and/or the IPG 101 may be configured such that the IPG 101 can use energy received from the external charger 200 for at least some of the IPG's operations (e.g., providing stimulation and/or providing power to at least some of its components) without first storing the energy in a rechargeable battery. In some embodiments, the external charger 200 is not configured to transmit communications to the IPG 101, and the external charger 200 may (or may not) be configured to receive communications from the IPG 101. For example, in some embodiments, the external charger 200 does not include a communications transmitter, and the external charger 200 has (or, in some such embodiments, does not have) a communications receiver. In some other embodiments, the external charger is configured to transmit communications to the IPG 101. For example, the external charger 200 may include a communications transmitter or transceiver.

The stimulation system 100 may further include one or more controller devices configured to control (e.g., wirelessly control) at least some of the operations of the IPG 101. For example, the controller device(s) may be communicatively coupled (e.g., wirelessly communicatively coupled via Bluetooth™, Bluetooth Low Energy, other protocols with suitable authentication and encryption to protect patient data, etc.) to the IPG 101. In some embodiments, the one or more controller devices are separate (e.g., physically separated) from the external charger 200. In the depicted embodiment, the stimulation system 100 includes a clinician programmer 302 and a patient remote 301 configured to control at least some of the operations of the IPG 101. The controller device(s) may be authorized to access and/or control the IPG 101. In some embodiments, the IPG 101 is configured to determine whether the controller device(s) are authorized to access and/or control the IPG 101 by any suitable means, such as cryptography (e.g., using private and/or public keys), when the controller device(s) communicate with the IPG 101. For example, a controller device authorized to access and/or control may send to the IPG 101 a communication encrypted using the controller device's private key, and the IPG 101 may determine that the controller device is an authorized device if the IPG 101 is able to decrypt the encrypted communication using the public key of the controller device.

Referring to FIG. 1B, the IPG 101 may include a processor 107; a memory device 108, for example, a non-volatile memory device (e.g., flash memory, or read-only memory (ROM), such as programmable read-only memory (PROM) or erasable programmable read-only memory (EPROM)); a communications device 109 (e.g., a receiver, a transmitter, and/or a transceiver); and/or a power supply 110 (e.g., a primary battery and/or a rechargeable battery). The communications device 109 may be configured to provide wireless communication links (e.g., through the skin of a person in whom the IPG 101 is implanted) to the clinician programmer device 302 and/or to the Patient remote 301. Wireless links may include Bluetooth, Bluetooth Low Energy, or other protocols with suitable authentication and encryption to protect patient data. In some embodiments, the memory device 108, the communications device 109, and/or the power supply 110 are communicatively coupled to each other through the processor 107. For example, the memory device 108, the communications device 109, and/or the power supply 110 may each be communicatively coupled to the processor 107. The processor 107, the memory device 108, the communications device 109, and/or the power supply 110 may be contained in the housing 102, and the proximal ends of the one or more electrical leads 103 may be electrically coupled to the power supply 110. In some embodiments, the IPG 101 includes additional components, such as a timer (e.g., a real-time clock (RTC)) configured to measure the passage of time, a driver configured to drive an electric current to the electrode 104, and/or a microcontroller coupled to the driver and configured to control the driver's driving of the electric current to the electrode 104. In some embodiments, the microcontroller may be included in the processor 107.

As used herein, the terms “processor” may include any combination of hardware, firmware, memory and software, employed to process data or digital signals. The hardware of a controller may include, for example, a microcontroller, application specific integrated circuits (ASICs), general purpose or special purpose central processors (CPUs), digital signal processors (DSPs), graphics processors (GPUs), and programmable logic devices such as field programmable gate arrays (FPGAs). In a processor, as utilized herein, each function is performed either by hardware configured (e.g., hard-wired) to perform that function, or by more general purpose hardware, such as a CPU, configured to execute instructions stored in a non-transitory storage medium or memory. A processor may contain two or more processors, for example, a processor may include two processors, an FPG and a CPU, interconnected on a PCB.

Referring to FIG. 1C, the IPG 101 may include a battery 120 (e.g., a rechargeable battery), a battery charger 122 (e.g., a lithium-ion battery charger), a voltage regulator 130, microcontroller 140, and a communication circuit 150. In some embodiments, the battery 120, the battery charger 122, and/or the voltage regulator 130 are included in the power supply 110; the microcontroller 140 is included in the processor 107; and/or the communication circuit 150 is included in the communications device 109.

The microcontroller 140 may control at least some operations of the IPG 101 and may be electrically coupled to, and configured to receive power from, the battery 120 along a first path 161. In some embodiments, the microcontroller 140 is electrically coupled to the first path 161 via a power port VDD. The first path 161 may include a first battery switch 146 that is configured to change between an opened configuration and a closed configuration. The opened configurations of the first battery switch 146 may be a configuration where the first path 161 is electrically disconnected at the first battery switch 146, and the closed configuration of the first battery switch 146 may be a configuration where the first path 161 is electrically connected at the first battery switch 146 (e.g., at least two portions of the first path 161 respectively on two sides of the first battery switch 146 may be electrically connected through the first battery switch 146). In some embodiments, the microcontroller 140 is configured to receive power from the battery 120 only along the first path 161, and the microcontroller 140 may be unable to receive power from the battery 120 when the first battery switch 146 is in the opened configuration. The first battery switch 146 may include any suitable type of switch, for example, one or more electronic switches and/or one or more mechanical switches (e.g., pole throw switch). For example, the first battery switch 146 may include a solid-state switch, such as a transistor (e.g., a MOSFET transistor, a relay transistor, etc.).

The communication circuit 150 may be configured to receive and/or transmit communications (e.g., wireless communications). For example, the communication circuit 150 may include a communications receiver, a communications transmitter, and/or a communications transceiver. The communication circuit 150 may be electrically coupled to, and configured to receive power from, the battery 120 along at least part of the first path 161. For example, the communication circuit 150 may be electrically coupled to the first path 161 via a power port VDD. In some embodiments, the communication circuit 150 is configured to receive power from the battery 120 only through at least part of the first path 161, and the communication circuit 150 may be unable to receive power from the battery 120 when the first battery switch 146 is in the opened configuration. In some embodiments, the communication circuit 150, in response to receiving power from the battery 120, is configured to advertise over a telemetry channel and to allow external devices (e.g., only external devices authorized to access and/or control the IPG 101) to connect with the IPG 101.

The voltage regulator 130 may be positioned along the first path 161. For example, the voltage regulator 130 may be electrically coupled between the first battery switch 146 and each of the microcontroller 140 and the communication circuit 150. The voltage regulator 130 may be configured to control or regulate a voltage received from the battery 120 and provided to various components of the IPG 101 (e.g., the microcontroller 140, the communication circuit 150, etc.). In some embodiments, the voltage regulator 130 is electrically coupled to a ground voltage 118.

The IPG 101 may be in a shelf mode when at least the microcontroller 140 is unable to receive power from the battery 120, for example, when the first battery switch 146 is in the opened configuration, and the IPG 101 may be in a normal mode when at least the microcontroller 140 is able to receive power from the battery 120, for example, when the first battery switch 146 is in the closed configuration. When the IPG 101 is in the normal mode, at least some components of the IPG 101 may consume power received from the battery 120, thereby causing the voltage or capacity of the battery 120 to drop over time. When the IPG 101 is in the shelf mode, the battery 120 can maintain its voltage or capacity above a minimum threshold value for a longer period of time (e.g., indefinitely) compared to when the IPG 101 is in the normal mode. If the voltage or capacity of the battery 120 falls below the minimum threshold value, it may be impossible, difficult, and/or dangerous to try to recharge the battery 120. As one example, charging the battery 120 after the voltage or capacity of the battery 120 has dropped below the minimum threshold value can strain the battery 120 and prematurely wear down the battery 120 and reduce the battery's lifecycle by reducing the number of charge cycles that the battery 120 can go through before it becomes non-operational. It may therefore be desirable for the IPG 101 to be in the shelf mode during long periods of non-use, for example, between the time that the IPG 101 is manufactured and the time that it is received by a retailer or sold to a customer.

The battery charger 122 may be electrically coupled to, and configured to receive power from, the battery 120 along a second path 162. The second path 162 may include a second battery switch 148 that is configured to change between an opened configuration and a closed configuration. The opened configuration of the second battery switch 148 may be a configuration where the second path 162 is electrically disconnected at the second battery switch 148, and the closed configuration of the second battery switch 148 may be a configuration where the second path 162 is electrically connected at the first battery switch 146 (e.g., at least two portions of the second path 162 respectively on two sides of the second battery switch 148 may be electrically connected). In some embodiments, the battery charger 122 is configured to receiver power from the battery 120 only along the second path 162, and the battery charger 122 may be unable to receive power from the battery 120 when the second battery switch 148 is in the opened configuration. The second battery switch 148 may include any suitable type of switch, for example, one or more electronic switches and/or one or more mechanical switches. For example, the first battery switch 146 may include a solid-state switch, such as a transistor. In some embodiments, the IPG 101 may be in the shelf mode when both the first battery switch 146 and the second battery switch 148 are in their respective opened configurations.

In some embodiments, part of the second path 162 may correspond to part of the first path 161. For example, a first part of the second path 162 and a first part of the first path 161 may each branch off from a common path, and at least part of the common path may define a second part of the second path 162 and a second part of the first path 161. In some other embodiments, the first and second paths 161 and 162 may be entirely separate from each other and may be separately electrically coupled to the battery 120 (e.g., at different ports of the battery 120).

The battery charger 122 may be electrically coupled to a receiver coil 124 configured to generate an electric current (e.g., an alternating electric current) in response to being in proximity with a changing magnetic field. The battery charger 122 may utilize the electric current generated by the receiver coil 124 to charge the battery 120. The battery charger 122 may provide power to the battery 120 along electrical paths other than the second path 162, and these other electrical paths have not been illustrated for convenience of illustration. At least some of the operations of the battery charger 122 may require power, and the battery charger 122 may use power received from the battery 120 along the second path 162 to power such operations.

The receiver coil 124 may include a core and a conductive wire wrapped around the core. The core of the receiver coil 124 may include a magnetic material, such as ferrite. Two ends of the receiver coil 124 (e.g., two ends of the conductive wire of the receiver coil 124 wrapped around the core of the receiver coil 124) may be respectively electrically coupled to two ports of the battery charger 122 so that the battery charger 122 may receive at least part of the electric current generated by the receiver coil 124. In some embodiments, one of the two ports of the battery charger 122 is an AC current receiver port ACIN, and the other one of the two ports of the battery charger 122 is a ground port GND. One of the two ends of the receiver coil 124 (e.g., the end electrically coupled to the ground port GND) may be electrically coupled to a ground voltage 115. In some embodiments, a first capacitor 126 (e.g., a variable capacitor) is electrically coupled between the two ends of the receiver coil 124.

The IPG 101 may be configured, in response to being positioned in a changing magnetic field (e.g., in proximity with a source of the changing magnetic field, for example, positioned at a position where a ratio of a magnitude of the magnetic field created by the source at the position to a maximum magnitude of the magnetic field created by the source is about 0.5 or more, about 0.6 or more, about 0.7 or more, about 0.8 or more, about 0.9 or more, about 0.95 or more, about 0.97 or more, or about 0.99 or more), to change at least one of the first battery switch 146 or the second battery switch 148 from their respective opened configurations to their respective closed configurations. For example, the receiver coil 124 may be configured, in response to being positioned in the changing magnetic field, to generate an electric current (e.g., an alternating electric current) and to change, via the electric current, at least one of the first battery switch 146 or the second battery switch 148 from their respective opened configurations to their respective closed configurations.

In some embodiments, the first and second battery switches 146 and 148 may be configured to change between their respective opened and closed configurations based on a change in electric current provided to respective control inputs (e.g., gate electrodes in some embodiments in which the first and second battery switches 146 and 148 respectively include a transistor) of the first and second battery switches 146 and 148. For example, the first battery switch 146 may include a transistor of a first type or kind that is configured to be off (e.g., in the opened configuration) when a voltage or electric current is not provided to a gate electrode of the transistor and to be on (e.g., in the closed configuration) when a voltage or electric current is provided to the gate electrode, or the first battery switch 146 may include a transistor of a second type or kind that is configured to be off (e.g., in the opened configuration) when a voltage or electric current is provided to a gate electrode of the transistor and to be on (e.g., in the closed configuration) when a voltage or electric current is not provided to its gate electrode.

In some embodiments, the first battery switch 146 may include a transistor of the first type, and the receiver coil 124 may be configured, in response to being in a changing magnetic field, to generate an electric current and to cause, via the generated electric current, a voltage or an electric current to be provided to the gate electrode of the transistor to turn on the transistor. For example, the IPG 101 may be configured so that at least part of the electric current generated by the receiver coil 124 can be provided to the gate electrode of the transistor to turn on the transistor.

In some other embodiments, the first battery switch 146 may include a first transistor of the second type, and the receiver coil 124 may be configured, in response to being in a changing magnetic field, to generate an electric current and to cause, via the generated electric current, a voltage or an electric current to cease being provided to the gate electrode of the first transistor. For example, the gate electrode of the first transistor may be electrically coupled to a power source (e.g., the battery 120 or another power source) that provides a voltage to the gate electrode of the first transistor to keep the first transistor turned off, and at least part of the electric current generated by the receiver coil 124 may be provided to a gate electrode of a second transistor of the second type electrically coupled between the power source and the gate electrode of the first transistor to cause the second transistor to turn off and cease to provide the voltage from the power source to the gate electrode of the first transistor.

The second battery switch 148 may include a transistor of the first type or of the second type, and the receiver coil 124 may be configured, in response to being in a changing magnetic field, to cause the transistor of the first type or the transistor of the second type to be turned on. The IPG 101 may be configured so that the receiver coil 124 can turn on the transistor of the second battery switch 148 in any manner that the receiver coil 124 can turn on a transistor of the first battery switch 146, and the IPG 101 may be configured so that receiver coil 124 can turn on the transistor of the second battery switch 148 in a same or different manner that the receiver coil 124 can turn on the transistor of the first battery switch 146.

In some embodiments, an end of the receiver coil 124 (e.g., the end of the receiver coil 124 electrically coupled to the AC current receiver port ACIN) is electrically coupled to the control input of the first battery switch 146 along a third path 163. The receiver coil 124 may be configured to provide at least part of an electric current generated by the receiver coil 124 to the input of the of the first battery switch 146 to change the first battery switch 146 from the opened configuration to the closed configuration. The third path 163 may include at least one rectifier device configured to at least partially rectify (e.g., half rectify or fully rectify) an alternating electric current generated by the receiver coil 124. For example, the rectifier device may be configured to receive the alternating electric current and provide a direct electric current. In the depicted embodiment, the third path 163 includes a first diode 132 (e.g., a Zener diode) configured to half rectify the alternating electric current generated by the receiver coil 124. In some other examples, a plurality (e.g., four) diodes may be utilized to fully rectify the alternating electric current.

A second capacitor 128 may be electrically coupled (e.g., coupled in parallel) to the third path 163 to at least partially smooth out (e.g., make substantially uniform in amplitude) the at least partially rectified electric current. A first electrode of the second capacitor 128 may be electrically coupled to the third path 163 (e.g., to a node along the third path 163), and a second electrode of the second capacitor 128 may be electrically coupled to a ground voltage 116. The third path 163 may include a second diode 134 (e.g., a Zener diode) electrically coupled between the first diode 132 and the control input of the first battery switch 146. The second diode 134 may prevent at least some electric current from flowing across the second diode 134 in a direction along the third path 163 from the second diode 134 toward the first diode 132.

In some embodiments, an end of the receiver coil 124 (e.g., the end of the receiver coil 124 electrically coupled to the AC current receiver port ACIN) is electrically coupled to a control input of the second battery switch 148 along a fourth path 164. The receiver coil 124 may be configured to provide at least part of an electric current generated by the receiver coil 124 to the input of the second battery switch to change the second battery switch 148 from the opened configuration to the closed configuration. The fourth path 164 may include at least one rectifier device configured to at least partially rectify (e.g., half rectify or fully rectify) an alternating electric current generated by the receiver coil 124. In the depicted embodiment, the fourth path 164 includes the first diode 132 configured to half rectify the alternating electric current generated by the receiver coil 124.

The second capacitor 128 may be electrically coupled (e.g., coupled in parallel) to the fourth path 164 (e.g., to a node along the fourth path 164) to at least partially smooth out the at least partially rectified electric current. The fourth path 164 may include one or more resistors between the rectifying device and the second battery switch 148 and that are configured to at least partially define a threshold amplitude that the at least partially rectified electric current must exceed to change the second battery switch 148 from the opened configuration to the closed configuration. In the depicted example, the fourth path 164 includes a first resistor 142 electrically coupled between the first diode 132 and the second battery switch 148. A second resistor 144 may be electrically coupled (e.g., coupled in parallel) to at least part of the fourth path 164. The first and second resistors 142, 144 may be connected in series. In the depicted example, the second resistor 144 is electrically coupled between a node on the fourth path 164 and a ground voltage 117. The second resistor 144, together with the first resistor 142, may at least partially define the threshold amplitude.

In some embodiments, at least part of the fourth path 164 may correspond to at least part of the third path 163. In the depicted example, a first part of the fourth path 164 and a first part of the third path 163 branch off from a common path that defines a second part of the third path 163 and a second part of the fourth path 164. In some embodiments, the third and fourth paths 163 and 164 include separate rectifying devices respectively configured to at least partially rectify the alternating current generated by the receiver coil 124. In some embodiments, one capacitor is electrically coupled to the third path 163 and configured to at least partially smooth out the at least partially rectified electric current provided by the rectifying device of the third path 163, and another capacitor is electrically coupled to the fourth path 164 and configured to at least partially smooth out the at least partially rectified electric current provided by the rectifying device of the fourth path 164.

The microcontroller 140 may be configured to cause the first battery switch 146 to be in the closed configuration. For example, the microcontroller 140 may be configured to transmit a first maintaining signal and to maintain, via the first maintaining signal, the first battery switch 146 in the closed configuration. In some embodiments, the IPG 101 may be configured such that at least part of the first maintaining signal transmitted by the microcontroller 140 is provided to the control input of the first battery switch 146 to maintain the first battery switch 146 in the closed configuration. For example, the control input of the first battery switch 146 may be electrically coupled to the microcontroller 140 (e.g., to an output port GP01 of the microcontroller 140) along a fifth path 165. The fifth path 165 may include a third diode 136 configured to at least partially prevent or block an electric current from flowing across the third diode 136 in a direction along the fifth path 165 from the third diode 136 toward the microcontroller 140.

The fifth path 165 and the third path 163 may be electrically coupled to the same control input (or to different control inputs) of the first battery switch 146. In some embodiments, the fifth path 165 and the third path 163 are electrically coupled to each other. For example, a portion of the third path 163 electrically coupled to the control input of the first battery switch 146 may correspond to a portion of the fifth path 165 electrically coupled to the control input of the first battery switch 146. The third diode 136 may at least partially prevent or block an electric current generated by the receiver coil 124 from flowing through the third diode 136 along the fifth path 165 to the microcontroller 140, and the second diode 134 may at least partially prevent or block an electric current generated by the microcontroller (e.g., the first maintaining signal) from flowing through the second diode 134 along the third path 163.

The communication circuit 150 may be configured to cause the first battery switch 146 to be in the closed configuration. For example, the communication circuit 150 may be configured to transmit a second maintaining signal and to maintain, via the second maintaining signal, the first battery switch 146 in the closed configuration. In some embodiments, the IPG 101 may be configured such that at least part of the second maintaining signal transmitted by the communication circuit 150 is provided to the control input of the first battery switch 146 to maintain the first battery switch 146 in the closed configuration. For example, the control input of the first battery switch 146 may be electrically coupled to the communication circuit 150 along a sixth path 166.

As explained above, when the IPG 101 is in the shelf mode, the first battery switch 146 is in the opened configuration so that at least some of the components (e.g., the microcontroller 140, the communication circuit 150, etc.) of the IPG 101 are unable to receive power from the battery 120. The IPG 101 may be configured to place itself into the shelf mode in response to receiving a telemetry command to do so. For example, when the IPG 101 is in the normal mode, the microcontroller 140 may terminate the first maintaining signal and/or the communication circuit 150 may terminate the second maintaining signal in response to receiving a telemetry command from an authorized controller device communicatively coupled to the IPG 101. Thus, for example, after the IPG 101 is manufactured, tested in normal mode, and determined to be suitable for use, the IPG 101 may be placed in shelf mode before being shipped from the manufacturer. The IPG 101 may be configured to be brought out of the shelf mode into the normal mode in response to being positioned in a changing magnetic field (e.g., in response to being positioned in proximity with a source of the changing magnetic field, for example, positioned at a position where a ratio of a magnitude of the magnetic field created by the source at the position to a maximum magnitude of the magnetic field created by the source is about 0.5 or more, about 0.6 or more, about 0.7 or more, about 0.8 or more, about 0.9 or more, about 0.95 or more, about 0.97 or more, or about 0.99 or more).

The external charger 200 may be configured to charge or power the IPG 101 via induction by creating a charge field (e.g., a changing magnetic field) around the external charger 200 and positioning the IPG 101 and the external charger 200 in proximity with each other (e.g., within inductive link range). For example, the external charger 200 may include one or more transmitting coils configured to generate an alternating magnetic field around the external charger 200 when the one or more transmitting coils are driven with respective AC currents. The changing magnetic field of the external charger 200 may therefore be utilized to bring the IPG 101 out of the shelf mode. By configuring the IPG 101 so that the external charger 200 is able to bring the IPG 101 out of the shelf mode, the need for a separate magnetic device designed to be used to bring the IPG 101 out of the shelf mode by conventional means is eliminated.

As explained above, the microcontroller 140 and/or the communication circuit 150 may be configured to maintain the first battery switch 146 in the closed configuration. In some embodiments, the microcontroller 140 and/or the communication circuit 150 may be configured, in response to the first battery switch 146 being changed from the opened configuration to the closed configuration so that power is provided from the battery 120 to the microcontroller 140 and/or to the communication circuit 150, to maintain the first battery switch 146 in the closed configuration. For example, in response to receiving power from the battery 120 along the first path 161, the microcontroller 140 may execute first computer-readable instructions stored in the memory device 108 to cause the microcontroller 140 to transmit a first maintaining signal and to maintain, via the first maintaining signal, the first battery switch 146 in the closed configuration. The microcontroller 140 may therefore be configured to maintain its supply of power from the battery 120 after the IPG 101 is taken out of the shelf mode.

The IPG 101 may be configured such that one selected from among the microcontroller 140 and the communication circuit 150 is configured to maintain the first battery switch 146 in the closed configuration and such that, in response to the one selected from among the microcontroller 140 and the communication circuit 150 ceasing to maintain the first battery switch 146 in the closed configuration (e.g., other than to put the IPG 101 in the shelf mode and/or in response to one or more conditions being satisfied), another one selected from the microcontroller 140 and the communication circuit 150 may maintain the first battery switch 146 in the closed configuration.

For example, the one selected from the microcontroller 140 and the communication circuit 150 may be the microcontroller 140, and the communication circuit 150 may be configured to maintain (e.g., via the second maintaining signal) the first battery switch 146 in the closed configuration in response to the microcontroller 140 being in a state (e.g., while being reset or reprogrammed) where the microcontroller 140 is unable to (or prevented from or set not to) maintain the first battery switch 146 in the closed configuration. In some other embodiments, the one selected from the microcontroller 140 and the communication circuit 150 may be the communication circuit 150, and the microcontroller 140 may be configured to maintain (e.g., via the first maintaining signal) the first battery switch 146 in the closed configuration in response to the communication circuit 150 being in a state (e.g., while being reset or reprogrammed) where the communication circuit 150 is unable to (or prevented from or set not to) maintain the first battery switch 146 in the closed configuration. In some embodiments, the communication circuit 150 is configured to maintain the first battery switch via a radio link.

The microcontroller 140 and the communication circuit 150 may be communicatively coupled such that the microcontroller 140 is configured to transmit communications to the communication circuit 150 and/or the communication circuit 150 is configured to transmit communications to the microcontroller 140. In some embodiments, the one selected from the microcontroller 140 and the communication circuit 150 may transmit a communication signal to the other one selected from the microcontroller 140 and the communication circuit 150 prior to the one selected from the microcontroller 1440 and the communication circuit 150 ceasing to maintain the first battery switch 146 in the closed configuration. The other one selected from the microcontroller 140 and the communication circuit 150 may be configured, in response to receiving the communication signal, to maintain the first battery switch 146 in the closed configuration.

The IPG 101 (e.g., the microcontroller 140 of the IPG 101) may be configured to perform a verification test in response to the IPG 101 being taken out of the shelf mode into the normal mode, and to return the IPG 101 to the shelf mode in response to the verification test not being satisfied or to maintain the IPG 101 in the normal mode if the verification test is satisfied. The verification test can prevent the IPG 101 from being unintentionally or inadvertently taken out of shelf mode prior to when the IPG 101 is intended to be taken out of the shelf mode. When the IPG 101 is unintentionally taken out of the shelf mode, the battery 120 may be drained below the minimum threshold value (as described above) before the IPG 101 is ready to be used. For example, many devices other than the external charger 200 may create changing magnetic fields that, when the IPG 101 is in proximity with one such device, may cause the first battery switch 146 to change from the opened configuration to the closed configuration to thereby unintentionally bring the IPG 101 out of the shelf mode. In such situations, the verification test may be executed, and the IPG 101 may return to the shelf mode in response to determining (e.g., by the microcontroller 140) that the IPG 101 was unintentionally taken out of the shelf mode.

In some embodiments, the IPG 101 may be configured, in response to the first battery switch 146 being changed from the opened configuration to the closed configuration, to maintain (e.g., by the microcontroller 140) the first battery switch 146 in the closed configuration for a set time period. The IPG 101 may be configured to cease to maintain (e.g., by the microcontroller 140) the first battery switch 146 in the closed configuration in response to set conditions not being satisfied within the set time period, or to continue to maintain (e.g., by the microcontroller 140) the first battery switch 146 in the closed configuration after the set time period if the set conditions are satisfied within the set time period. The conditions may include receiving by the IPG 101 (e.g., by the communication circuit 150) a communication (e.g., a request to access and/or control at least some operations of the IPG 101) from an external device. In some embodiments, the set time period may be about 60 minutes, about 45 minutes, about 30 minutes, about 15 minutes, about 10 minutes, about 5 minutes, about 3 minutes, or about 1 minute.

In some embodiments, the set conditions may include receiving a communication from an external device that is authorized to access and/or control the IPG 101. Requiring the external device to be an authorized device may provide additional protection against the IPG 101 being unintentionally taken out of the shelf mode and/or accessed or controlled by unauthorized external devices that are configured to transmit communications in the same or similar bandwidth as the IPG 101 is configured to receive. In some embodiments, the controller device(s) (e.g., the patient remote 301 and/or the clinician programmer 302) may be authorized to access and/or control the IPG 101. Accordingly, the IPG 101 may be taken out of the shelf mode and into the normal mode by positioning the IPG 101 and the external charger 200 in proximity to each other and using the external charger 200 to generate a changing magnetic field, and the IPG 101 may be maintained in the normal mode by communicating, via the patient remote 301 or the clinician programmer 302, with the IPG 101 within the set time period after bringing the IPG 101 out of the shelf mode.

For example, in some embodiments, the microcontroller 140 may, in response to receiving power from the battery 120 after the first battery switch 146 is closed, execute the first computer-readable instructions stored in the memory device 108 to initiate a timer configured to end after the set time period and to transmit a first maintaining signal to maintain, via the first maintaining signal, the first battery switch 146 in the closed configuration.

The microcontroller 140 may, in response to the IPG 101 not receiving any communication from an external device within the set time period, execute second computer-readable instructions stored in the memory device 108 to terminate the first maintaining signal transmitted to the first battery switch 146 to cease to prevent, via the first maintaining signal, the first battery switch 146 from changing from the closed configuration to the opened configuration. For example, the communication circuit 150 may be configured to receive communications from external devices and to send a confirmation signal (e.g., the received communication itself or a signal indicating that the communication was received) to the microcontroller 140, and the microcontroller 140 may, in response to receiving the confirmation signal and via its execution of the second computer-readable instructions stored in the memory device 108, terminate the first maintaining signal transmitted to the first battery switch 146.

The microcontroller 140 may, in response to the IPG 101 receiving a communication from the external device within the set time period, execute third computer-readable instructions stored in the memory device 108 to continue to transmit the first maintaining signal to maintain, via the first maintaining signal, the first battery switch 146 in the closed configuration. In some embodiments, the microcontroller 140 may, via execution of the third computer-readable instructions, determine whether the external device is authorized to access and/or control the IPG 101. For example, the IPG 101 may have public keys corresponding to external devices authorized to access and/or control the IPG 101 stored in the memory device 108, and the microcontroller 140 may determine whether the communication received from the external device can be decrypted using any of the stored public keys. In response to being unsuccessful in decrypting the received communication, the microcontroller 140 may determine that the external device is unauthorized to access and/or control the IPG 101. In response to successfully decrypting the received communication, the microcontroller 140 may determine that the external device is authorized to access and/or control the IPG 101.

The microcontroller 140 may, in response to determining that the external device is authorized to access and/or control the IPG 101, and via execution of the third computer-readable instructions, continue to transmit the first maintaining signal and to maintain, via the first maintaining signal, the first battery switch 146 in the closed configuration after the set time period lapses, thereby maintaining the IPG 101 in the normal mode. The microcontroller 140 may, via execution of the third computer-readable instructions and in response to (i) the microcontroller 140 determining that the external device is not authorized to access and/or control the IPG 101 and (ii) the IPG 101 not otherwise receiving any communications from an authorized external device within the set time period, cease to transmit the first maintaining signal.

For each of the operations of the microcontroller 140 described herein, the memory device 108 of the IPG 101 may store instructions, which, when executed by the microcontroller 140, cause the microcontroller 140 to perform those operations.

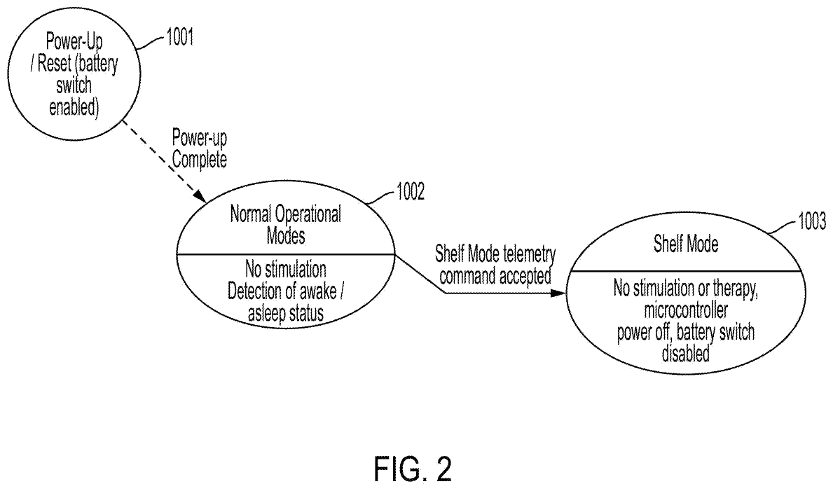

FIG. 2 is a shelf mode method flow diagram for placing an electronic device in a shelf mode. The shelf mode method of FIG. 2 may be used with any electronic device within the scope of the present disclosure, such as an IPG. Referring to FIG. 2, the shelf mode method may include a first process (or task) 1001 of powering up or resetting an electronic device. For example, the first process 1001 may begin when the first battery switch of the electronic device is changed from the opened configuration to the closed configuration, or when the electronic device is turned on.

The shelf mode method may include a second process (or task) 1002 of performing operations in a normal mode of the electronic device, such as providing treatment (e.g., stimulation treatment), charging an internal battery of the electronic device, etc. During the normal mode, the electronic device may determine (e.g., by a microcontroller of the electronic device) whether the electronic device is in an awake mode or in a sleep mode. In the sleep mode, certain functions of the electronic device that are performed when the electronic device is in the awake mode may be disabled. The electronic device may change from the awake mode to the sleep mode if certain conditions (e.g., providing treatment, such as stimulation) are not satisfied within a set time period. For example, the microcontroller may determine whether stimulation has been provided within the set time period. If no stimulation has been provided within the set time period, the microcontroller may change the electronic device from being in the awake mode to being in the sleep mode.

The shelf mode method may include a third process (or task) 1003 of placing (e.g., by the microcontroller) the electronic device in the shelf mode. In some embodiments, the electronic device may (e.g., by the microcontroller) change from the normal mode to the shelf mode in response to being in the sleep mode, or in response to not being taken out of the sleep mode into the awake mode within a set time period. In some embodiments, the electronic device may change from the normal mode to the shelf mode in response to the electronic device (e.g., the microcontroller) receiving and verifying a telemetry command to go into the shelf mode. For example, the telemetry command may be transmitted by an external device authorized to control the electronic device. In response to receiving and verifying the telemetry command, the microcontroller may output a logic level to disable a battery switch electrically coupling an internal battery of the electronic device with at least the microcontroller to cause the electronic device to go into the shelf mode.

In some embodiments, the electronic device, in response to the microcontroller ceasing to maintain the electronic device in the normal mode and one or more conditions being satisfied (e.g., the microcontroller being reset or reprogrammed), may be configured to maintain itself in the normal mode other than via the microcontroller. For example, the electronic device may include a communications circuit and may maintain itself in the normal mode via a communications circuit in response to the microcontroller ceasing to maintain the electronic device in the normal mode, for example, in response to the microcontroller being unable to (or prevented from or set not to) maintain the electronic device in the normal mode. In some embodiments, the microcontroller may transmit a communication signal to the communication circuit, and the communication circuit may maintain the electronic device in the normal mode in response to receiving the signal from the microcontroller. The electronic device may therefore be able to continue operations in the normal mode even when the microcontroller ceases to maintain the electronic device in the normal mode.

The electronic device may include a memory device and, for each of the operations of the microcontroller described herein, the memory device may have stored instructions that, when executed by the microcontroller, cause the microcontroller to perform such operations.

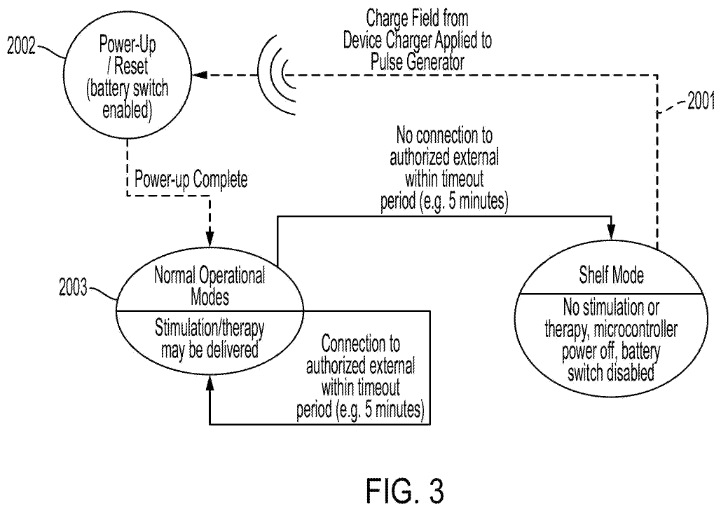

FIG. 3 is a normal mode method flow diagram for bringing an electronic device out of the shelf mode. The normal mode method may be used with any electronic device within the scope of the present disclosure, such as an IPG. Referring to FIG. 3, the normal mode method may begin with the electronic device being in the shelf mode, wherein a microcontroller of the electronic device is disconnected from a battery of the electronic device. For example, a first battery switch electrically coupled between the microcontroller and the battery may be in an opened configuration wherein the microcontroller is electrically disconnected from the battery at the first battery switch. At least some of the functions of the electronic device may be disabled when the electronic device is in the shelf mode. For example, the electronic device may be an implantable pulse generator configured to provide stimulation to a person or living being, and the implantable pulse generator may be unable to provide stimulation when the electronic device is in the shelf mode.

The normal mode method may include a first process (or task) 2001 of applying a charge field (e.g., a changing magnetic field) from a charger device to the electronic device to electrically connect the microcontroller to the battery, for example, by inducing the first battery switch to change from the opened configuration to a closed configuration wherein the first battery switch electrically connects two adjacent portions of an electrical path coupling the microcontroller and the battery.

The normal mode method may include a second process (or task) 2002 of powering up or resetting the electronic device after power has been re-established between the microcontroller and the battery.

The normal mode method may include a third process (or task) 2003 of waiting, for a set timeout period (e.g., about five minutes), to receive a request for connection from an authorized external device. If the electronic device receives no connection request within the set timeout period, or if the electronic device receives only one or more connection requests from nonauthorized external devices (e.g., external devices that the electronic device does not recognize and/or that are not authorized to access and/or control the electronic device) within the set timeout period, then the electronic device may return (e.g., by the microcontroller) to the shelf mode. However, if the electronic device receives a connection request and determines (e.g., by the microcontroller) that the electronic device is an authorized external device within the set timeout period, then the electronic device may connect to the authorized external device and stay in the normal mode after the set timeout period expires. During the normal mode, the electronic device may perform at least some of its operations. For example, the electronic device may be the implantable pulse generator, and the implantable pulse generator may be configured to provide stimulation when in the normal mode.

The electronic device may include a memory device and, for each of the operations of the microcontroller described herein, the memory device may have stored instructions that, when executed by the microcontroller, cause the microcontroller to perform such operations.

Non-limiting and non-exhaustive examples of electronic devices, systems including the electronic device, and methods for putting the electronic device in a shelf mode and bringing the electronic device out of the shelf mode have been described herein. It will be understood that features described with respect to one or more embodiments may be included in one or more other embodiments. The scope of the present disclosure is defined by appended claims and equivalents thereof.

What is claimed is:

1. An electronic device, comprising:
a battery;
a microcontroller configured to receive power from the battery along a first path comprising a first battery switch configured to change between an opened configuration and a closed configuration; and
a receiver coil configured, in response to being positioned in a changing magnetic field, to generate an alternating electric current and to change, via the alternating electric current, the first battery switch from the opened configuration to the closed configuration.

2. The electronic device of claim 1, wherein the receiver coil is electrically coupled to the first battery switch along a second path comprising a rectifying device configured to at least partially rectify the alternating electric current and to provide a direct current to the first battery switch.

3. The electronic device of claim 1, further comprising a battery charger electrically coupled to the receiver coil and configured to receive power from the battery along a third path comprising a second battery switch configured to be changed between an opened configuration and a closed configuration,
wherein the receiver coil is configured to change, via the alternating electric current, the second battery switch from the opened configuration to the closed configuration.

4. The electronic device of claim 3, wherein the receiver coil is electrically coupled to the second battery switch along a fourth path comprising a rectifying device configured to at least partially rectify the alternating electric current and to provide a direct current to the second battery switch.

5. The electronic device of claim 4, wherein the fourth path further comprises one or more resistors between the rectifying device and the second battery switch and to at least partially define a threshold amplitude that the direct current must exceed to change the second battery switch from the opened configuration to the closed configuration.

6. The electronic device of claim 1, wherein the microcontroller is configured to transmit a first maintaining signal and to maintain, via the first maintaining signal, the first battery switch in the closed configuration.

7. The electronic device of claim 6, wherein the microcontroller is electrically coupled to the first battery switch along a fifth path and is configured to provide the first maintaining signal to the first battery switch.

8. The electronic device of claim 6, wherein the microcontroller is configured to controllably terminate the first maintaining signal to cease to maintain, via the first maintaining signal, the first battery switch in the closed configuration.

9. The electronic device of claim 6, further comprising a communication circuit configured to transmit a second maintaining signal and to maintain, via the second maintaining signal, the first battery switch in the closed configuration.

10. The electronic device of claim 9, wherein the communication circuit is configured to transmit the second maintaining signal in response to the microcontroller ceasing to transmit the first maintaining signal and one or more set conditions being satisfied.

11. The electronic device of claim 10, wherein the one or more set conditions comprise at least one of the microcontroller being reset or the microcontroller being reprogrammed.

12. The electronic device of claim 1, wherein the electronic device is configured, in response to the first battery switch being changed from the opened configuration to the closed configuration so that power is provided from the battery to the microcontroller, to:
maintain, by the microcontroller, the first battery switch in the closed configuration for a set time period, and
cease to maintain, by the microcontroller and in response to the electronic device not receiving a communication from an external device within the set time period, the first battery switch in the closed configuration.

13. The electronic device of claim 12, wherein the electronic device is configured to maintain, by the microcontroller and in response to receiving a communication from the external device within the set time period, the first battery switch in the closed configuration after the set time period.

14. The electronic device of claim 12, wherein the electronic device is configured, in response to receiving a communication from the external device within the set time period, to:
cease to maintain, by the microcontroller, the first battery switch in the closed configuration if the external device is not authorized to control the electronic device and the electronic device receives no communication within the set time period from an authorized external device, or
maintain, by the microcontroller, the first battery switch in the closed configuration after the set time period if the external device is authorized to control the electronic device.

15. The electronic device of claim 1, wherein the electronic device comprises an implantable pulse generator configured to provide stimulation to a tissue proximal to the electronic device.

16. A system, comprising:
an electronic device, comprising:
a battery, and
a microcontroller configured to receive power from the battery along a first path comprising a first battery switch configured to be changed between an opened configuration and a closed configuration; and
an external charger configured to wirelessly charge the battery and to change the first battery switch from the opened configuration to the closed configuration,
wherein the electronic device is configured, in response to the first battery switch being changed from the opened configuration to the closed configuration by the external charger such that power is provided from the battery to the microcontroller, to:
maintain, by the microcontroller, the first battery switch in the closed configuration for a set time period, and
cease to maintain, by the microcontroller and in response to not receiving a communication from an external device within the set time period, the first battery switch in the closed configuration.

17. The system of claim 16, wherein the electronic device is configured to maintain, by the microcontroller and in response to receiving a communication from the external device within the set time period, the first battery switch in the closed configuration after the set time period.

18. The system of claim 17, further comprising a communication circuit configured, in response to the microcontroller ceasing to maintain the first battery switch in the closed configuration at a time after the set time period lapses and in response to one or more conditions being satisfied, to maintain the first battery switch in the closed configuration.

19. The system of claim 17, wherein the electronic device is configured, in response to receiving a communication from the external device within the set time period, to:
cease to maintain, by the microcontroller, the first battery switch in the closed configuration if the external device is not authorized to control the electronic device and the electronic device receives no communication within the set time period from an authorized external device, or
maintain, by the microcontroller, the first battery switch in the closed configuration after the set time period if the external device is authorized to control the electronic device.

20. The system of claim 17, wherein the external charger is configured to generate a changing magnetic field that, in response to the electronic device being positioned in proximity with the external charger, causes the first battery switch to change from the opened configuration to the closed configuration.
